(19) United States
(12) Patent Application Publication
(10) Pub. No.: US 2022/0228000 A1
(43) Pub. Date: Jul. 21, 2022

(54) STABILIZATION OF PHYCOCYANINS

(71) Applicant: CHR. HANSEN NATURAL COLORS A/S, Hoersholm (DK)

(72) Inventors: JEAN-THOMAS PIERSON, Arpajon (FR); CARINE MANE, Arpajon (FR); KLAUS KOEHLER, Hoersholm (DK); LAURA GOURY, Arpajon (FR); VINCENT MAZUREK, Arpajon (FR); VINCENT MAZUREK, Arpajon (FR)

(21) Appl. No.: 17/614,616

(22) PCT Filed: May 28, 2020
(86) PCT No.: PCT/EP2020/064845

§ 371 Date: Nov. 29, 2021

(30) Foreign Application Priority Data
May 29, 2019 (EP) 19177303.5

(51) Int. Cl. C09B 61/00; C09B 67/02; A23L 5/46; A23L 2/58

(52) U.S. Cl. CPC C09B 61/00; C09B 67/0097; A23V 2002/00; A23L 2/58; A23L 5/46

(57) ABSTRACT

The present invention relates to a stabilized phycocyanin composition comprising a complexing agent and an encapsulating agent as stabilizer. The present invention further relates to a method for preparing the composition and the use of the composition for coloring particularly food products.

### FIELD OF THE INVENTION

The present invention relates to stabilized phycocyanin compositions comprising a complexing agent and an encapsulating agent. The present invention further relates to processes of producing said phycocyanin compositions and the use of said compositions for coloring food, feed, pet food, pet treats, cosmetics, nutrients and pharmaceuticals.

### BACKGROUND OF THE INVENTION

Phycocyanin is a pigment with a characteristic blue color. Phycocyanin is found in cyanobacterium also called blue-green algae. The blue phycocyanin pigment from cyanobacterium is often referred to as spirulina.

Phycocyanin is commonly isolated from spirulina algae and shows many dietary and therapeutic attributes. Therefore, spirulina and spirulina extracts have been used for many years as food or nutritional components. Phycocyanins are known to be very sensitive to temperature and pH changes in the environment, Seo et al., Int. J. Mol. Sci. 2013, 14, 1778-1787. Thus, there have been several attempts to stabilize phycocyanins for coloring purposes. WO15090697 describes the stabilization of phycocyanobilin with a polyphenol to obtain a stabilized complex by cleaving the phycocyanin either chemically or enzymatically and reacting with the polyphenol. WO18134390 describes the use of glycerol as stabilizer for phycocyanin. In Food Hydrocolloids, January 2018 74:46-52, Selig et al. describes the enhancement of color stability of spirulina based phycocyanin via a polysaccharide complex.

CN108684819 relates to a fruit and vegetable dairy beverage comprising vegetable extract of spirulina, osmanthus fruit pigment and banana peel polyphenol extract. Where the banana peel polyphenol is believed to have antioxidant effect on the human body and help to eliminate fatigue. Spirulina is added for its amount of proteins.

US2019059422 relates to a pelletized colorant which is a combination of spirulina called a pigment protein complex and an encapsulating agent. No complexing agent is added to the composition.

Despite these attempts to find a good solution, the problem still exists and there is still a need to find a suitable solution to stabilizing phycocyanins.

### SUMMARY OF THE INVENTION

It is therefore the aim of the present invention to provide a stabilized phycocyanin coloring composition and said stabilized composition comprises a phycocyanin, a complexing agent and an encapsulating agent.

The present inventors have unexpectedly found that by precipitating phycocyanin with a complexing agent and an encapsulating agent it is possible to obtain a stable colorant composition.

A first aspect of the present invention relates to a coloring composition comprising a phycocyanin, a complexing agent and an encapsulating agent. In particular wherein the complexing agent is a polyphenol and the encapsulating agent is selected from the group consisting of lecithins, citric acid esters, glycerol ester of fatty acids, monoglycerides, diglycerides, triglycerides, ethoxylated monoglyceride ester of fatty acids, polyglycerol ester of fatty acids, glycerol ester of fatty acids, sorbitan esters of fatty acids, sucrose esters of fatty acids, polysorbates, quillaja extracts, caseinate, beet pectins, starches, starch derivatives, starch octenyl succinate derivatives, guar gum, acacia gum, gum arabic, xanthan gum and any mixtures thereof.

A second aspect of the present invention relates to a process for the preparation of the coloring composition according to the first aspect, comprising the steps of:
1a) blending a phycocyanin with a complexing agent in an aqueous solution to obtain a precipitate;
1b) separating the precipitate of 1a) from the supernatant;
1c) adding an encapsulating agent; and
1d) homogenizing the formulation
or
2a) blending a phycocyanin with an encapsulating agent in an aqueous solution;
2b) homogenizing the formulation of 2a);
2c) adding a complexing agent; and
2d) optionally centrifuging the formulation of 2c)
or
3a) blending a complexing agent with an encapsulating agent in an aqueous solution;
3b) homogenizing the formulation of 3a);
3c) adding a phycocyanin; and
3d) optionally centrifuging the formulation of 3c)

A third aspect of the present invention relates to a method of coloring a food or a feed, a pet food, a pet treat, a nutrient and/or a pharmaceutical with a coloring composition comprising a phycocyanin, a complexing agent and an encapsulating agent.

A fourth aspect of the present invention is directed to a food product, an animal feed, a pet food, a pet treat, a cosmetic, a nutrient and/or a pharmaceutical product comprising the composition according to the first aspect of the present invention.

A fifth aspect of the present invention relates to use of the coloring composition according to the first aspect of the present invention as a colorant for food, animal feed, pet food, pet treats, cosmetics, nutrients and/or pharmaceuticals.

### DETAILED DESCRIPTION OF THE INVENTION

By coloring composition is meant a composition which purpose when added to a product is to provide color to the product it is added to.

The present invention is directed to a stabilized coloring composition comprising a phycocyanin, a complexing agent and an encapsulating agent.

It has surprisingly been found that precipitation of phycocyanin with a complexing agent in the presence of an encapsulating agent result in a stable composition. which allows for retention of the blue color especially under acid conditions such as in for example soft drinks.

In a particular embodiment the phycocyanin is obtained from the cyanobacteria Arthrospira platensis. The phycocyanin is preferably derived from spirulina. In a particular embodiment of the present invention the phycocyanin is food grade or pharmaceutically acceptable coloring agents.

The phycocyanin is preferably extracted from cyanobacteria such as but not limited to Arthrospira platensis. Normally such extract is comprising at least 2% (w/w), such as at least 5% (w/w), such as at least 10% (w/w), such as at least 20% (w/w), such as at least 30% (w/w), such as at 40% (w/w), such as at least 50% (w/w) of phycocyanin.

In a preferred embodiment of the present invention the amount of phycocyanin present in the composition is at least 0.5% (w/w), such as at least 0,7% (w/w), such as at least 0.9% (w/w), such as at least 1% (w/w), such as at least 1.5% (w/w), such as at least 2% (w/w), such as at least 2.5% (w/w), such as at least 3% (w/w).

In a preferred embodiment of the present invention the amount of phycocyanin present in the composition is less than 30% (w/w), such as less than 25% (w/w), such as less than 20% (w/w) such as less than 15% (w/w), such as less than 13% (w/w), such as less than 10% (w/w), such as less than 9% (w/w), such as less than 8% (w/w), such as less than 7% (w/w), such as less than 6% (w/w), such as less than 5% (w/w), such as less than 4% (w/w), such as less than 3% (w/w).

In a particular embodiment of the present invention the amount of phycocyanin present in the composition is between 0.5 to 30% (w/w), such as between 0.5 to 20% (w/w), such as between 0.5 to 12% (w/w), such as between 0.5 and 5% (w/w), such as between 0.8 and 3% (w/w), such as between 1 to 20% (w/w), such as between 2 to 12% (w/w), such as between 3 to 5 % (w/w).

The complexing agent of the present invention may be any compound capable of forming a complex with phycocyanin. In a particular embodiment the complex obtained is obtainable or is obtained without cleaving off the phycocyanobilin from the phycocyanin. In a particular embodiment of the present invention the phycocyanin is not cleaved.

In a preferred embodiment of the present invention the complexing agent is a polyphenol. In a particular embodiment of the present invention the polyphenol is selected from the group consisting of hydrolizable tannins and gallotannins such as tannic acid, polygalloylglucoses, gallic acid, flavanols such as catechine, epicatechine, epigallocatechine, epigallocatechine gallate, phenolic acids such as 4-hydroxybenzoic acid, rosmarinic acid and cinnamic acid digallic acid, condensed tannins (condensation products of flavans), quercitannic acid, Gallo tannic acid, quercitin, and ellagitannins, or natural extracts containing those polyphenols. In a preferred embodiment the polyphenol is tannic acid and/or gallic acid.

In a preferred embodiment of the present invention the amount of complexing agent present in the composition is at least 0.005% (w/w), such as at least 0.01% (w/w), such as at least 0.02% (w/w).

In a preferred embodiment of the present invention the amount of complexing agent such as the polyphenol is present in the composition is less than 10% (w/w), such as less than 9% (w/w), such as less than 8% (w/w), such as less than 7% (w/w), such as less than 6% (w/w), such as at least 5% (w/w).

In a particular embodiment of the present invention the amount of complexing agent in the composition is between 0.005 to 10% (w/w), such as between 0.01 to 8% (w/w) such as between 0.02 to 5% (w/w), such as between 0.5 to 5% (w/w).

The ratio (w/w) of phycocyanin to complexing agent may be in the range of 1:20 to 20:1, such as 1:10 to 10:1, such as 1:3 to 3:1.

The ratio of phycocyanin to complexing agent may also be reported as weight of phycocyanin to one mol of complexing agent. In a particular embodiment of the present invention the ratio between mass of phycocyanin to one mol of complexing agent is in the range of 10:1 to 10000:1, such as in the range of 50:1 to 6000:1. In a preferred embodiment of the invention the ratio between mass of phycocyanin to one mol of complexing agent is in the range of 70:1 to 500:1.

The encapsulating agent of the present invention may be an emulsifier, a hydrocolloid or a thickener such as natural gums.

The emulsifier of the present invention may be but are not limited to lecithins, citric acid esters, glycerol ester of fatty acids, monoglycerides, diglycerides, triglycerides, ethoxylated monoglycerides, polyglycerol ester of fatty acids, glycerol ester of fatty acids, sorbitan esters of fatty acids, sucrose esters of fatty acids, polysorbates, quillaja extracts, caseinate and mixtures thereof.

Polysorbates are oily liquids derived from ethoxylated sorbitan (a derivative of sorbitol) esterified with fatty acids. Common brand names for polysorbates include Scattics, Alkest, Canarcel, and Tween.

The polysorbate may e.g. be polysorbate 20, polysorbate 40, polysorbate 60, polysorbate 65, polysorbate 80 or a mixture thereof. In a particular embodiment of the present invention the polysorbate is polysorbate 80.

Suitable examples of polysorbate include polysorbate with E-numbers: E 431, E 432, E433, E 434, E 435 or E 436.

The hydrocolloids of the present invention may be but are not limited to polysaccharides such as beet pectins, starch, starch derivatives such as starch octenyl succinate derivative and any mixtures thereof

The gums of the present invention may be but are not limited to natural gums such as guar gum, acacia gum, gum arabic, xanthan gum and any mixtures thereof.

In a preferred embodiment the gum is guar gum. In a more preferred embodiment the guar gum is in a combination with gallic acid and spirulina.

In a preferred embodiment of the present invention the amount of encapsulating agent present in the composition is at least 0.1% (w/w), such as at least 0.2% (w/w), such as at least 0.3% (w/w), such as at least 0.4% (w/w), such as at least 0.5% (w/w).

In a preferred embodiment of the present invention the amount of encapsulating agent present in the composition is less than 40% (w/w), such as less than 30% (w/w), such as less than 25% (w/w), such as less than 22% (w/w), such as less than 21% (w/w), such as less than 20% (w/w).

In a particular embodiment of the present invention the amount of encapsulating agent of the composition is between 0.1 to 40% (w/w), such as between 0.2 to 30% (w/w), such as between, such as between 0.25 to 25% (w/w), 0.3 to 25 (w/w), such as between 0.5 to 20% (w/w).

The nature of the encapsulating agent has an impact on the amount used in the present formulation.

In a preferred embodiment of the present invention the amount of emulsifier or hydrocolloid present in the composition is at least 5% (w/w), such as at least 6% (w/w), such as at least 7% (w/w), such as at least 8% (w/w), such as at least 10% (w/w).

In a preferred embodiment of the present invention the amount of emulsifier or hydrocolloid present in the composition is less than 40% (w/w), such as less than 30% (w/w), such as less than 25% (w/w), such as less than 22% (w/w), such as less than 21% (w/w), such as less than 20% (w/w).

In a particular embodiment of the present invention the amount of emulsifier or hydrocolloid of the composition is between 5 and 40% (w/w), such as between 7 to 30%, such as between 10 to 20% (w/w).

In a preferred embodiment of the present invention the amount of gum present in the composition is at least 0.1% (w/w), such as at least 0.15% (w/w), such as at least 0.2% (w/w), such as at least 0.25% (w/w).

In a preferred embodiment of the present invention the amount of gum present in the composition is less than 2% (w/w), such as less than 1% (w/w), such as less than 0.9% (w/w), such as less than 0.8% (w/w), such as less than 0.7% (w/w),

In a particular embodiment of the present invention the amount of gum of the composition is between 0.1 to 2% (w/w), such as between 0.15 to 1% (w/w).

There are several ways to classify the color of a substance. Generally, the color characteristics of a substance may be characterized by three parameters, the hue, the saturation and the lightness. Hue is an attribute associated with each of the dominant wavelengths of the visible spectrum and reflects the dominant color of the composition (red, yellow, blue etc.). Chroma is the saturation and pertains to the intensity of the color composition often described as the vividness or the dullness of a color. Lightness reflects the amount of white or black in the color composition. In a particular embodiment of the present invention color is measured by the use of a spectrometer or a similar instrument capable of taking readings on hue, chroma and lightness in accordance with CIE (Commission Internationale de L'Eclairage—the International Commission of Illumination) tristimulus system: CIELCH. In CIELCH system, L* denotes lightness, C* specifies chroma and h denotes the hue.

The composition of the present invention is particularly useful as a colorant. In a particular embodiment the composition has a blue color defined by a hue value between 180° and 280° , such as between 200° and 250° when measured with a spectrometer such as a Datacolor 650 (Datacolor) or a Probe4Light (Pleiades).

The composition may be used in blends with other pigments, such as carotenes, and especially with β-carotene or carthamus to form green colors, and with anthocyanins or betalains to form purple colors.

In a particular embodiment the invention covers blends of the present composition with other pigments, said blend having a purple color defined by a hue value of 240° to 300° when measured on a spectrometer such as a Datacolor 650 (Datacolor) or a Probe4Light (Pleiades).

In another particular embodiment the invention covers blends of the present composition with a carotenoid resulting in a green color defined by a hue value of 70° to 140° when measured on a spectrometer such as a Datacolor 650 (Datacolor) or a Probe4Light (Pleiades).

In a particular embodiment of the present invention the composition is considered having improved stability if DE2000 of the formulated product is lower than the DE2000 of the spirulina blue extract raw material when measured in a soft drink at pH 3. This stability is evaluated through measuring the LCh values of a bottle containing soft drink at pH 3, with the composition at TO and after 1 week. A DE2000 value is calculated from the LCh values of the composition at both TO and T1 week.

The present invention is further related to a process of preparing a composition of the present invention.

In a particular embodiment of the present invention the process for preparation of the composition of the present invention, comprises the steps of:
1a) blending a phycocyanin with a complexing agent in an aqueous solution to obtain a precipitate;
1b) separating the precipitate of 1a) from the supernatant;
1c) adding an encapsulating agent; and
1d) homogenizing the formulation
or
2a) blending a phycocyanin with an encapsulating agent in an aqueous solution;
2b) homogenizing the formulation of 2a);
2c) adding a complexing agent; and
2d) optionally centrifuging the formulation of 2c)
or
3a) blending a complexing agent with an encapsulating agent in an aqueous solution;
3b) homogenizing the formulation of 3a);
3c) adding a phycocyanin; and
3d) optionally centrifuging the formulation of 3c)

In a preferred embodiment the complexing agent is a polyphenol and the encapsulating agent is selected from the group consisting of lecithins, citric acid esters, glycerol ester of fatty acids, monoglycerides, diglycerides, triglycerides, ethoxylated monoglyceride ester of fatty acids, polyglycerol ester of fatty acids, glycerol ester of fatty acids, sorbitan esters of fatty acids, sucrose esters of fatty acids, polysorbates, quillaja extracts, caseinate, beet pectins, starches, starch derivatives, starch octenyl succinate derivatives, guar gum, acacia gum, gum arabic, xanthan gum and any mixtures thereof.

The separation step in method 1 may be performed by any suitable method. In a particular embodiment of the present invention the separation is performed by centrifugation or filtration such as by plaque filtration, microfiltration or ultrafiltration.

The homogenization step may be performed by any suitable method such as vigorous stirring, by use of a high shear mixer, by use of a homogenizer or by milling.

In a particular embodiment of the present invention the particle size is between 0.1 μm and 1000 μm. Such as between 0.1 μm to 100 μm, such as between 0.1 μm to 10 μm.

The composition of the present invention may find use as colorant for products such as food products, animal feeds, pet food, pet treats, cosmetics, nutrients and pharmaceutical products.

In a preferred embodiment the food product is selected from the group consisting of a beverage such as soft drinks, flavored water, fruit juices, punches, concentrated forms of these beverages but also alcoholic beverages and instant beverage powders, confectionary products, wine gum, cake, cookies, dessert, candies, puddings, jellies, instant pudding powder, marmalade, jam, sugar confectionary, panned chocolate lentils, ice cream, cereals, but also in snacks, sausage casings, pasta, macaroni, cheese, milk drinks or yoghurt, soy milk, salad dressing, soups, sauces, dairy fruit prep, prepared food and/or extruded food.

In one embodiment cosmetic preparations are selected from cream, tooth paste, makeup and dermal products.

In a further embodiment, pharmaceutical preparations are selected from unguents, pills, tablets and capsules.

The coloring composition of the present invention may be added to the product either as an aqueous stock solution or a pre-blend with other suitable ingredients according to the specific application. The coloring composition may be added to the product in a coating such as for chocolate lentils.

The coloring composition is particularly useful in low pH applications such as in application where pH of the product is below 7, such as below 6, such as below 5, such as below 4, or even below a pH of 3.

Most food products are in the acidic range and a specific useful application includes coloring of beverage products. In beverage products the pH is typically around 2 to 6 and the beverage product is therefore preferably a beverage product, wherein the pH is from pH 2 to 6.

The following items are preferred embodiments of the present invention:

Item 1. A composition comprising a phycocyanin, a complexing agent and an encapsulating agent.

Item 2. The composition according to item 1, wherein it is a coloring composition.

Item 3. The composition according to any of the preceding items, wherein the composition is an aqueous solution.

Item 4. The composition according to any of the preceding items, wherein the phycocyanin and the complexing agent is forming a complex.

Item 5. The complex according to item 4, comprising a phycocyanin and a complexing agent obtainable by mixing the phycocyanin and the complexing agent in an aqueous solution.

Item 6. The composition according to any of the preceding items, wherein the complexing agent is a polyphenol.

Item 7. The composition according to item 6, wherein the polyphenol is selected from the group consisting of tannins, flavanols, phenolic acids and polyphenol rich natural extracts such as white grape extracts and grape seed extracts.

Item 8. The composition according to any of the preceding items wherein the phycocyanin is originating from algae.

Item 9. The composition according to item 8, wherein the algae is spirulina.

Item 10 The composition according to any of the preceding items, wherein the color hue of the composition is between 180° and 280°.

Item 11. The composition according to any of the preceding items, wherein the color hue of the composition is between 200° and 250°.

Item 12. The composition according to any of the preceding items, wherein the composition is blended with a second pigment to obtain a more green or purple color.

Item 13. The composition according to item 12, wherein the color hue of the composition is between 240° to 300°.

Item 14. The composition according to item 12, wherein the color hue of the composition is between 70° to 140°.

Item 15. The composition according to any of the preceding items, wherein the encapsulating agent is selected from the group consisting of emulsifiers, hydrocolloids and thickeners.

Item 16. The composition according to item 15, wherein the emulsifier is selected from the group consisting of lecithins, citric acid esters, glycerol ester of fatty acids, monoglycerides, diglycerides, triglycerides, ethoxylated monoglycerides, polyglycerol ester of fatty acids, glycerol ester of fatty acids, sorbitan esters of fatty acids, sucrose esters of fatty acids, polysorbates, quillaja extracts, caseinate and any mixtures thereof.

Item 17. The composition according to item 15, wherein the hydrocolloid is selected from the group consisting of beet pectins, starches, starch derivatives, starch octenyl succinate derivatives and any mixtures thereof.

Item 18. The composition according to item 15, wherein the thickener is a natural gum which is selected from the group consisting of guar gum, acacia gum, gum arabic, xanthan gum and any mixtures thereof.

Item 19. A process for preparation of the composition according to any of items 1 to 18, comprising the steps of:
1a) blending a phycocyanin with a complexing agent in an aqueous solution to obtain a precipitate;
1b) separating the precipitate of 1a) from the supernatant;
1c) adding an encapsulating agent; and
1d) homogenizing the formulation
or
2a) blending a phycocyanin with an encapsulating agent in an aqueous solution;
2b) homogenizing the formulation of 2a);
2c) adding a complexing agent; and
2d) optionally centrifuging the formulation of 2c)
or
3a) blending a complexing agent with an encapsulating agent in an aqueous solution;
3b) homogenizing the formulation of 3a);
3c) adding a phycocyanin; and
3d) optionally centrifuging the formulation of 3c)

Item 20. A food product, a feed product, a pet food, a pet food treat, a cosmetic, a nutrient or a pharmaceutical product comprising the colorant composition according to any of items 1 to 18.

Item 21. The food product according to item 20, wherein the food product is beverage such as soft drinks, flavored water, fruit juices, punches concentrated forms of these beverages but also alcoholic beverages and instant beverage powders, confectionary products, wine gum, cake, cookies, dessert, candies, puddings, jellies, instant pudding powder, marmalade, jam, sugar confectionary, panned chocolate lentils, ice cream, cereals, but also in snacks, sausage casings, pasta, macaroni, cheese, milk drinks or yoghurt, soy milk, salad dressing, soups, sauces, dairy fruit prep, prepared food and/or extruded food.

Item 22. Use of the composition according to any of the items 1 to 18 in food, feed, pet food pet treat, cosmetic, nutrient and pharmaceuticals.

Item 23. Use of the composition according to any of the items 1 to 18 as a colorant.

Embodiments of the present invention are described below, by way of non-limiting examples.

### EXAMPLES

### Example 1

5.3g of carrier free spirulina blue extract powder was dissolved into 100 mL of distilled water. 2.82 g of gallic acid was dissolved into 100 mL of distilled water at 45° C. The gallic acid solution was slowly poured into the spirulina blue extract solution under stirring and let to complex overnight. The final solution was centrifuged (4000 RPM for 30 minutes) to obtain 28 g of sediment. 0.775 g of guar gum was dispersed into 110 mL of distilled water. The sediment containing blue spirulina extract complexed with the gallic acid was then mixed with the guar gum solution.

The solution was high shear mixed using a Silverson laboratory high shear mixer (2 minutes at mid speed) to obtain a homogenous solution. This supernatant was tested in a soft drink media (pH 3 solution) at a 6 g/L dosage. The solution was measured on a spectrometer (Probe4Light from Pleiades) for evaluation of the LCh parameters. Reference was the raw spirulina blue extract sample in soft drink media prepared at 0.6 g/L dosage to obtain a visual match of the formulated product dosage. Bottles containing the formulated colorant and the reference sample were measured both at TO and after 1 week for evaluation of the difference of LCh parameters after 1 week storage in the dark at ambient temperature.

TABLE 1
LCh Colorimetric parameters measured on
formulated colorant and reference sample.

| Sample | L | C | h | DE2000 |
|---|---|---|---|---|
| Reference T0 | 30 | 66 | 283 | — |
| Reference 1 week | 37 | 61 | 270 | 9.77 |
| Formulated colorant 1 T0 | 43 | 61 | 265 | — |
| Formulated colorant 1 week | 45 | 56 | 253 | 6.31 |

After 1 week the LCh evaluation of bottles led to calculate the DE2000 for the reference at 9.77, which was 3.46 points more than the formulated colorant which had a DE2000 of 6.31. The formulated colorant showed improved stability performance under acid conditions such as soft drink media compared to unformulated raw spirulina blue extract.

### Example 2

21.6 g of carrier free spirulina blue extract powder was dissolved into 300 mL of distilled water. 80 g of polysorbate 80 was added under stirring to the previous solution after dissolution of the phycocyanin. The solution was strongly stirred to allow homogenous dispersion of the encapsulating agent. 8 g of tannic acid, as complexing agent, was dissolved in 50 mL of distilled water. The complexing agent solution was then added under stirring to the blue extract formulated with polysorbate 80 and kept under stirring until the solution was homogenous. The final solution was centrifuged to obtain a dark blue homogenous supernatant. This supernatant was tested in soft drink media (pH 3 solution) at a 6 g/L dosage. The solution was measured on a spectrometer for evaluation of the LCh parameters. Reference was the raw spirulina blue extract sample in soft drink media prepared 0.6 g/L dosage to obtain a visual match of the formulated product dosage. Bottles containing the formulated colorant and the reference sample were measured at TO and after 1 week for evaluation of the difference of LCh parameters after 1 week storage in the dark at ambient temperature.

TABLE 2
LCh Colorimetric parameters measured on
formulated colorant and reference sample.

| Sample | L | C | h | DE2000 |
|---|---|---|---|---|
| Reference T0 | 30 | 66 | 283 | — |
| Reference 1 week | 37 | 61 | 270 | 9.77 |
| Formulated colorant T0 | 38 | 69 | 280 | — |
| Formulated colorant 1 week | 42 | 62 | 272 | 5.97 |

The LCh evaluation of the DE2000 for the reference was 9.77, which was 3.80 points more than the formulated colorant which had a DE2000 of 5.97. The formulated colorant showed improved stability performance under acid conditions such as soft drink media compared to unformulated raw spirulina blue extract.

1. A coloring composition comprising a phycocyanin, a polyphenol as complexing agent and an encapsulating agent and wherein the encapsulating agent is selected from the group consisting of lecithins, citric acid esters, glycerol ester of fatty acids, monoglycerides, diglycerides, triglycerides, ethoxylated monoglyceride ester of fatty acids, polyglycerol ester of fatty acids, glycerol ester of fatty acids, sorbitan esters of fatty acids, sucrose esters of fatty acids, polysorbates, quillaja extracts, caseinate, beet pectins, starches, starch derivatives, starch octenyl succinate derivatives, guar gum, acacia gum, gum arabic, xanthan gum and any mixtures thereof;

2. The composition according to claim 1, wherein the composition is an aqueous solution.

3. The composition according to claim 1, wherein the polyphenol is selected from the group consisting of tannins, flavanols, phenolic acids and polyphenol rich natural extracts such as white grape extracts or grape seed extracts.

4. The composition according to claim 1, wherein the phycocyanin is originating from algae.

5. The composition according to claim 4, wherein the algae is spirulina.

6. The composition according to claim 1, wherein the color hue of the composition is between 180° and 280°.

7. The composition according to claim 1, wherein the color hue of the composition is between 200° and 250°.

8. The composition according to claim 1, wherein the composition is blended with a second pigment to obtain a more green or purple color.

9. The composition according to claim 8, wherein the color hue of the composition is between 240° to 300°.

10. The composition according to claim 8, wherein the color hue of the composition is between 70° to 140°.

11. A process for preparation of the composition according to claim 1, comprising the steps of:
1a) blending the phycocyanin with the polyphenol in an aqueous solution to obtain a precipitate;
1b) separating the precipitate of 1a) from the supernatant;
1c) adding the encapsulating agent; and
1d) homogenizing the formulation
or
2a) blending the phycocyanin with the encapsulating agent in an aqueous solution;
2b) homogenizing the formulation of 2a);
2c) adding the polyphenol; and
2d) optionally centrifuging the formulation of 2c)
or
3a) blending the polyphenol with the encapsulating agent in an aqueous solution;
3b) homogenizing the formulation of 3a);
3c) adding the phycocyanin; and
3d) optionally centrifuging the formulation of 3c)

12. A food product, a feed product, a pet food, a pet food treat, a cosmetic, a nutrient or a pharmaceutical product comprising the colorant composition according to claim 1.

13. The food product according to claim 12, wherein the food product is beverage such as soft drinks, flavored water, fruit juices, punches concentrated forms of these beverages but also alcoholic beverages and instant beverage powders, confectionary products, wine gum, cake, cookies, dessert, candies, puddings, jellies, instant pudding powder, marmalade, jam, sugar confectionary, panned chocolate lentils, ice cream, cereals, but also in snacks, sausage casings, pasta, macaroni, cheese, milk drinks or yoghurt, soy milk, salad dressing, soups, sauces, dairy fruit prep, prepared food and/or extruded food.

14. Use of the composition according to claim 1 in food, feed, pet food, pet treat, cosmetics, nutrients and/or pharmaceuticals as a colorant.
